(19) United States
(12) Patent Application Publication
(10) Pub. No.: US 2004/0260358 A1
(43) Pub. Date: Dec. 23, 2004

(54) Triggered electromyographic test device and methods of use thereof

(76) Inventors: Robin Vaughan, San Diego, CA (US); Jean-Jacques Abitbol, San Diego, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 10/683,253

(22) Filed: Oct. 10, 2003

Related U.S. Application Data
(63) Continuation-in-part of application No. 10/463,266, filed on Jun. 17, 2003.

(51) Int. Cl. A61N 1/18

(52) U.S. Cl. 607/48

(57) ABSTRACT

The present invention provides a test device and apparatus comprising a triggered electromyographic stimulator (TES) for determining nerve proximity and status during surgical procedures, and methods of use thereof. The invention TES linked with a connector capable of connecting to a standard, commercially available EMG processing system. The corresponding neuromuscular response is detected to indicate the position of the TES adjacent to nerve proximity. The present invention provides relatively easy, flexible and convenient method for determining nerve proximity and status during the surgical procedure.

### RELATED APPLICATIONS

The present application is a continuation-in-part of application Ser. No. 10/463,266, filed on Jun. 17, 2003, entitled “Triggered Electromyographic Test Device and Methods of Use Thereof”, which is incorporated by reference herein in its entirety.

### TECHNICAL FIELD

The present invention relates to electrophysiology medical devices for nerve monitoring systems and methods during surgical procedure, and more particular, to an electromyographic (EMG) test device including triggered EMG stimulator (TES) for nerve proximity and status detection systems and methods.

### BACKGROUND

Avoiding unintentionally contacting a patient's nerves and causing damage to these nerves when performing surgical procedures is important. In particular, since spinal nerves control major body functions, avoiding inadvertent contact with these nerves is extremely important, but difficult due to the high density of these nerves in the spine region.

A variety of systems and methods for monitoring nerves and muscles during surgical procedures are available and described in the literature. Generally, these systems apply a current to a stimulator to evoke a muscular response and, require the user to observe the evoked muscular response by relying on visible muscles twitches. When such a muscular response is observed, the stimulator is considered to be near the nerve coupled to the responsive muscle. The observation of the muscular response is difficult depending on the competing tasks of the user, and the muscular response may be suppressed during general anesthesia, limiting the ability of a user to detect the response.

There are other limitations and problems related to the use of these existing systems and methods. For instance, the existing systems are incapable of determining the distance of the nerve to the stimulator or instrument passing through tissue or passing by the nerves, raising the specter of inadvertent contact with, and/or possible damage to, the nerves.

Electromyographic (EMG) monitoring systems have been employed to provide an electrical stimulation and monitor its response during the application of a stimulation signal to a nerve or nerve root, the pedicle screw and/or the pre-formed hole. Such applications are disclosed, for instance, in PCT applications WO 03/026482 and WO 03/005887, respectively. However, the system and methods of use thereof described in the above-identified PCT applications are only available when using the specifically designed EMG systems as described in the applications. Such specifically designed EMG systems are expensive, and require specialists to perform the procedure and to interpret the complicated data, limiting their availability and applicability.

Therefore, a need for an improved system and method that can determine the nerve proximity during the surgical procedures exists. Such system and method should be relatively easy and flexible to operate and should be commercially available, if possible.

### SUMMARY

The present invention provides test devices including at least one triggered electromyographic stimulator (TES) for nerve proximity detection during the surgical procedures. The TES includes at least three components: an electrode used to stimulate the nerves or nerve roots, pedicle screws, or pedicle screw holes during the surgical procedures; means to transmit electric signals from the EMG system to the electrode; and a connector capable of connecting to a standard electromyogram (EMG) processing system to process the electric signals for determination of nerve proximity and status.

In a first aspect, the present invention provides a triggered electromyographic test device contains a triggered EMG stimulator (TES). In one of the preferred embodiments, such TES comprises preferably a ball electrode at one terminus of the stimulator, although any appropriate electrode shape can be used. The ball tip electrode is preferably about 3.0 mm in diameter although any appropriate size can be used. In another preferred embodiment, the TES is substantially straight, having a length of about between about 6 inches to about 16 inches, although any appropriate length can be used. In yet another preferred embodiment, the TES is utilized to stimulate the nerves, nerve roots, or the interior of a hole formed in a pedicle or a pedicle screw after insertion into the hole.

In another aspect, the present invention provides that a TES contains a means to transmit signals. The TES stimulates the nerve, nerve roots, or pedicle screw inside the hole which is received by a standard, commercially available EMG processing system. Such EMG process system measures and processes EMG responses of muscle groups associated with a particular nerve. The changes of the EMG responses indicate that the nerve is considered to be near the surgical tools or the TES and, thus, the nerve proximity and status can be detected during surgical procedure.

In yet another aspect, the present invention provides that a TES contains a connector capable of connecting to any standard, commercially available EMG processing system. Moreover, the TES is designed for use percutaneously through a cannula or catheter, or in open surgical procedures.

In yet another aspect, the present invention provides methods of determining nerve proximity and status during surgical procedure and assessment using the TES of the present invention. Such methods include a stimulation of the nerve, nerve roots, or the interior of a hole formed in a pedicle or a pedicle screw using the TES. Since the present invention provides that the TES is connected with an EMG process system by a connector, the EMG system receives the responses to the electrical stimulation, which indicates the position of the nerve(s) in relation to the TES and the surgical tools used during the surgical procedure.

In yet another aspect, the present invention provides an apparatus for determining nerve proximity and status during surgical procedure and assessment. The invention apparatus comprises a test device including a TES and a standard EMG processing system. The TES stimulates the nerve, nerve roots or the interior of a hole formed in a pedicle or a pedicle screw to produce electrical signals. The EMG processing system delivers the electrical signals and processes the signals into neuromuscular responses. The apparatus is appropriate for determining the nerve proximity and status during the surgical procedure by identifying the relationship between the neuromuscular response and the electrical stimulation signals.

In yet another aspect, the present invention provides a triggered electromyographic test device containing a triggered EMG stimulator (TES). In one of the preferred embodiments, such TES comprises preferably a flush tip electrode at one terminus of the stimulator, although any appropriate electrode shape can be used. In another preferred embodiment, the test device is unitary and its components are not easily or readily detachable. In another preferred embodiment, the TES is made to be flexible such that the flush tip end of the TES can be engaged on a the surface of a tissue of interest, and a surgeon can put forward pressure at the other end of the TES in order to reversibly bend the TES in a desired configuration, and once the surgeon releases the forward pressure on the TES, the TES returns to its original substantially straight configuration.

### BRIEF DESCRIPTION OF THE DRAWINGS

FIG. 1 depicts one aspect of a triggered EMG test device 10 of the present invention. The test device 10 comprises a triggered EMG stimulator (TES) 11, illustrated as a ball-tip at one end; coupled with an insulated shaft (about 6 inches to about 16 inches) 12a, which is further linked to a shrink sleeve and a lead wire (about 60 inches) 12b; and a TES safety connector 13, capable of connecting to an EMG processing system 14. All elements are operably linked to conduct an electric signal.

FIG. 2 depicts one aspect of a triggered EMG test device 20 of the present invention. The test device 20 is flexible and is shown under forward pressure and against a surface in a reversibly bent configuration. The test device 20 comprises a triggered EMG stimulator (TES) 21, illustrated as substantially flush tip at one end; coupled with an insulated shaft (about 6 inches to about 16 inches) 22a, which is further linked to a shrink sleeve and a lead wire (about 60 inches) 22b; and a TES safety connector 23, capable of connecting to an EMG processing system 24. All elements are operably linked to conduct an electric signal and are not intended to be readily separable.

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Terms of orientation such as “up” and “down” or “upper” or “lower” and the like refer to orientation of the parts during use of the device. Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings:

Other technical terms used herein have their ordinary meaning in the art that they are used, as exemplified by a variety of technical dictionaries.

Introduction

The present invention recognizes that it can be desirable to have an easily operable triggered electromyographic test device for use during surgical procedure and assessment to avoid inadvertently contacting nerve(s) and to detect nerve proximity and status. The test device has preferably a long and substantially straight TES, preferably having a ball-tip electrode, or the like, at one terminus and a connector at another terminus in relation to the electrode. Preferably, the TES is linked to a connector capable of connecting to a standard EMG processing system. More preferable, the TES is able to stimulate a nerve or nerve root, pedicle screw, or pedicle screw hole, such stimulation is received and processed by a commercially available EMG processing system, indicating the position between the surgical tools and the particular nerves. The present invention provides such a device and methods of use.

As a non-limiting introduction to the breath of the present invention, the present invention includes several general and useful aspects, including:

1. the TES comprising an electrode; means to transmit electric signals; and a connector capable of connecting to a standard EMG processing system for detecting nerve proximity and status during surgical procedure;

2. the EMG processing system that is standard and commercially available; and

3. the TES that is used percutaneously through a cannula or a catheter, or in an open procedure;

4. the TES comprising an optionally flexible and substantially flush tip electrode; means to transmit electric signals; and a connector capable of connecting to a standard EMG processing system for detecting nerve proximity and status during surgical procedure; all of which form a unitary test device which is not intended to be readily separable.

These aspects of the invention, as well as others described herein, can be achieved by referring to the methods, articles of manufacture, compositions of matter, and illustrations depicted in the figures and as described herein. To gain a full appreciation of the scope of the present invention, it will be further recognized that various aspects of the present invention can be combined to make desirable embodiments of the invention.

I Triggered EMG Test Device

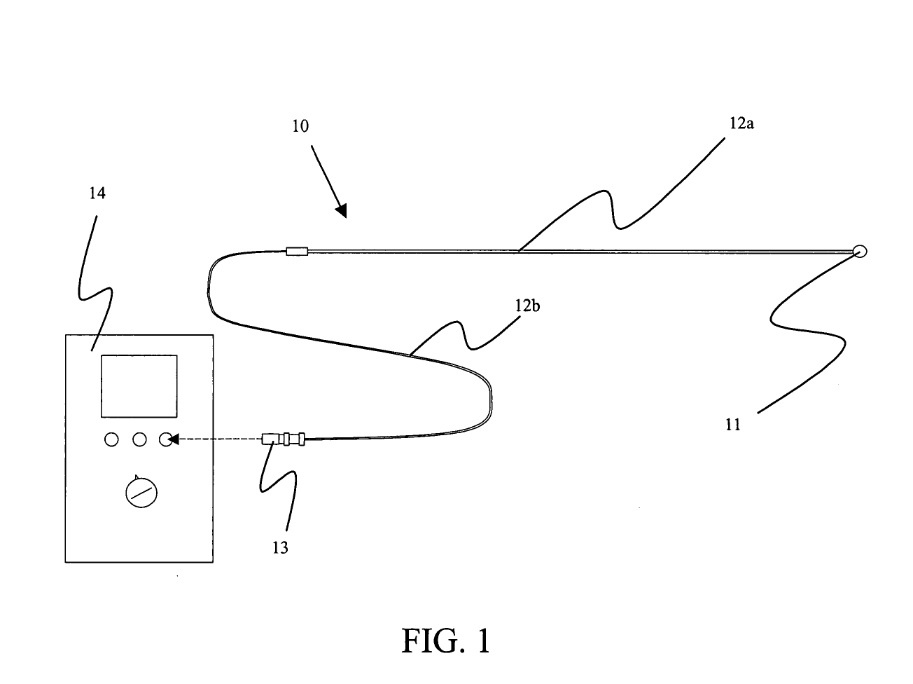

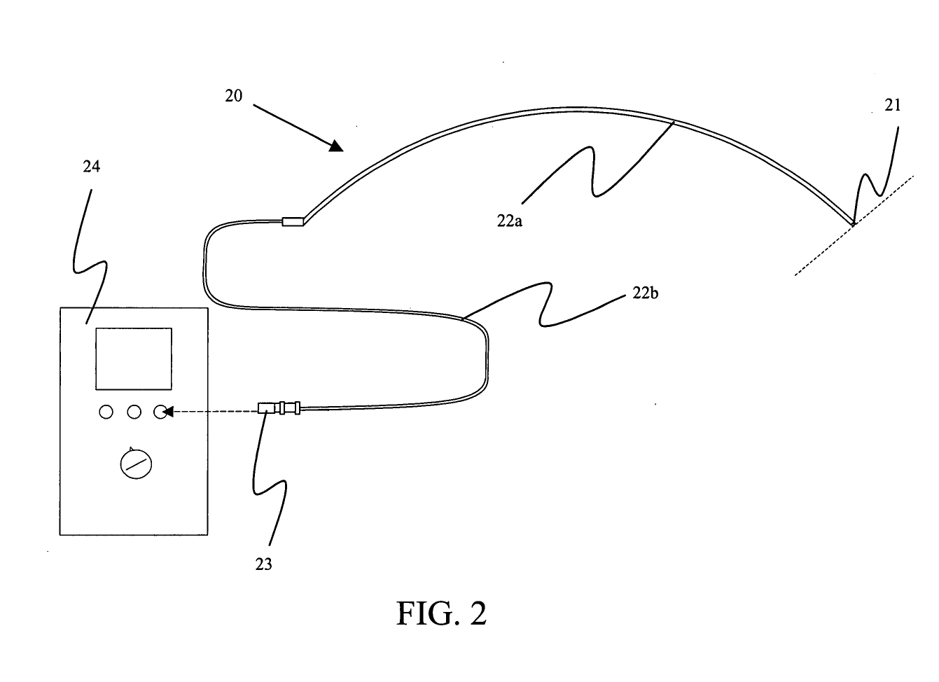

The present invention provides a triggered EMG test device including a TES. The TES includes an electrode (depicted in FIG. 1 as a ball end, and depicted in FIG. 2 as a substantially flush tip end, operably linked with a shaft), coupled with means (depicted as a shrink sleeve and a wire) to transmit signals, and a connector (depicted as a safety connector) capable of connecting to a standard EMG processing system for detecting nerve proximity and status during surgical procedure. In one of the embodiments, the TES is utilized to stimulate nerve or nerve roots, a pedicle screw or a pedicle screw hole, percutaneously through a cannula or catheter, or in an open surgical procedure.

THE TES ELECTRODE: The first element of the triggered EMG test device is the TES including an electrode at one terminus. The electrode transmits an electrical signal and therefore should be constructed from an electrically conductive material. In one of the embodiments, the electrode can be a ball or other shaped electrode. Preferably, such ball-tip electrode is about 3.0 mm in diameter, although any appropriate size can be used. In another of the embodiments, the electrode can be a substantially flush tip or other shaped electrode. Preferably, such ball-tip electrode is about 3.0 mm in diameter, and such flush-tip electrode is about 3.0 mm across, although any appropriate size can be used. The electrode is preferably made of silver, although other metals or semimetals, such as gold, platinum, alloy metals, and the like can also be used. The variety of electrode shapes include but not limited to square, triangle, disc shape, finger clip shape, bar shape, ring shape, ground shape, and other appropriate shapes. Moreover, the electrodes can be needle electrodes, such as coated or uncoated needle electrodes, or concentric needle electrodes in a wide range of sizes. For example, the electrodes can be coated or uncoated stainless steel monopolar needle electrodes. All the electrodes can be disposable or reusable, made of gold, silver, or other suitable metals.

MEANS TO TRANSMIT ELECTRIC SIGNALS: In yet another embodiment, the electrode of the TES operably linked to a substantially straight shaft being able to transmit an electric signal from the electrode to the connecting wire. In one embodiment, the TES is operably linked to a flexible shaft such that the tip end of the TES can be engaged on a the surface of a tissue of interest and a surgeon can put forward pressure at the other end of the TES in order to reversibly bend the TES in a desired configuration, and once the surgeon releases the forward pressure on the TES, the TES returns to its original substantially straight configuration. The shaft may be malleable or deformable, such that in certain surgical procedures, the surgeon is able to reversibly bend the insulating shaft and adapt the electrode to a desired shape and configuration. In another embodiment, the shaft can be non-reversibly flexible meaning that once the surgeon puts forward pressure on one end of the shaft, the shaft is deformed and remains in bent configuration even after the forward pressures is released from the shaft. Various lengths of the shaft can be used. For instance, the shaft preferably has an average length between about 1 inches to about 20 inches, preferably, between about 5 inches to about 18 inches, more preferably, between about 6 inches to about 16 inches, and most preferably, between about 10 inches to about 16 inches. In general, the smaller length of the stimulator is preferably used in an open surgical procedure, and the longer length of the stimulator is preferably used in a percutaneous surgical procedure. The shaft of the TES can be rigid, but preferably thin, semi-rigid, flexible, semi-flexible, or formable. It can be insulated in its entire length, part of the length, or substantially its entire length. For example, one of the materials used for insulation of the shaft is kynar insulation. Any conductive materials, such as metals or alloys, rubber, Teflon, plastic, kynar, silicon, can all be used as insulation, but the coating on the outer shaft can be material that does not conduct electricity such as rubber or polypropylene, or the like. In one of the preferred embodiments, the shaft is about 16 ga nickel silver, although other appropriate metals can also be used.

The shaft is further operably connected to a connecting wire or a cable with or without a shrink sleeve located opposite to the electrode end of the stimulator. In one of the preferred embodiments, the lead is connected to the shaft via a solder connection covered by shrink tubing. The connecting wire is in a variety of size. Preferably, the connecting wire can be about 1 to about 1.5 meters in length. The connecting wire is preferably made of lead, insulated and super-flexible. The cable length, the thickness of the cable, and the materials used for the cable can vary.

In one embodiment, the test device is substantially unitary and is operably linked to conduct an electric signal. The components of the substantially unitary device are connected together such that each component will not detach easily or are irreversibly attached. The means for connecting the components of the test device, either permanently or semi-permanently, can be any appropriate means depending on the material used for each component of test device. These attaching means include interlocking complementary surfaces, fusing, soldering, gluing, screwing, or any other means that will result in a test device with permanently or semi-permanently fixed components that are not intended to be readily separable. In one embodiment, the components are irreversibly snapped together.

THE CONNECTOR: Furthermore, the TES includes a connector linked to the connecting wire or cable, and appropriate for further connecting to a standard EMG processing system. The connector is preferably a standard 1.5 mm female connector, or known as a “DIN” universal connector. DIN is short for Deutsches Insitut für Normung eV (the standards-setting organization for Germany). A DIN connector is a connector that conforms to one of the many standards defined by DIN, are used widely in the skilled art.

EMG PROCESSING SYSTEM: Electromyography (EMG) is a procedure for measuring the electrical activity of muscles. When a muscle contracts a minute electrical charge is generated. This charge can be detected and recorded by a technologist, neurologist or neurophysiologist to determine the order in which specific muscles fire. This is referred to as a “motor action pattern”.

In general, the TES is connected to the EMG “output” section. Most EMG devices have several “output” ports and different programs employ different outputs for a variety of reasons. Most of these systems are programmable so identifying which port is employed is unnecessary. The system itself may deliver electrical stimulations ranging from 0.0 milliamps to 100 milliamps with variable rates of delivery and variable durations of stimulations. In one of the embodiments, the rate of delivery is preferably about 2.9/second and the durations of stimulation is preferably about 200 microseconds.

Any commercially available evoked potential system, such as Nicolet Viking, Cadwell Cascade, Biologic EMG “type” instruments, incorporates within it an EMG system, and suitable for use for the present invention. The EMG system portion of the evoked potential instruments employs electrodes that are placed in/on strategic muscle groups innervated by the nerves or nerve roots at risk during the surgery. These electrodes are placed into/onto the muscles themselves and connected to the amplifier portion of the EMG system. The ongoing electrical activity of the muscles is amplified, filtered, and displayed on a computer screen and appears as straight lines that are moving left to right. When a deviation of the electrical output from the muscles occurs that deviation is demonstrated on the screen as a positive or negative deflection from the flat baseline. An EMG is actually a polyphasic positive and negative deflection of a minimum of about 20 microvolt amplitude.

When a triggered EMG is performed, the EMG system monitors the ongoing electrical output of the muscle. The stimulation provided by the EMG system travels from the system via the output port into the stimulator (TES) and onto the nerve or nerve roots. When a sufficient intensity of stimulation is presented the nerve or nerve roots depolarizes, for example, sends the signal along, through the nerve into the muscle itself. The muscle then contracts and relaxes minutely or maximally depending on the amount of stimulation and the EMG system records that increase in electrical output of the muscle. A polyphasic response is observed from the baseline and an EMG has occurred.

In determination of nerve proximity, an electrical stimulation from the EMG system is presented to tissue, such as brain, muscle, ligament, bone, and the like. When a sufficient intensity of stimulation is presented that electrical stimulus transverses the tissue and finds its way to a nerve. That nerve then depolarizes and the muscle fires when the stimulation is high enough. Depending on the tissue being stimulated, the threshold that the nerve depolarizes and the muscle firs gives some indication of the distance between the stimulator and the nerve itself. Therefore, in a pedicle screw situation, if an EMG occurs with a threshold of about 3 milliamps, that is a strong indication that the pedicle wall has been breached and the electrical signal is traveling from the TES, through the pedicle, through the breached pedicle wall, into the nerve, causing the nerve to depolarize and the muscle to fire and the EMG system to pick up the deviation from the baseline after amplifying and filtering the ongoing electrical activity from the muscle itself.

II A Method of Detecting Nerve Proximity and Status Using the Triggered EMG Test Device

The methods disclosed herein may be performed using a variety of the TES test device configurations as previously described and as set forth in the disclosed methods and examples.

The present invention method of the “triggered EMG” technique using the TES test device is employed for identifying nerves or nerve roots, identifying which nerve roots fire which muscles for determination of nervous system integrity, testing pedicle screws in their holes, and testing the holes themselves if a pedicle wall breach is suspended.

In one of the preferred embodiments, a surgeon is holding a TES in his/her hands and placing it directly on tissue (for direct nerve or nerve proximity tests). The operator of the EMG system then begins to ramp up the intensity of the stimulation until an EMG is observed on the EMG system. That intensity threshold then is employed in determining if the stimulator is on nerve, near nerve, or on other material (for example a tumor). A similar application is where the surgeon again places the stimulator on the nerve in question and another threshold is obtained by the operator ramping up the stimulation intensity until an EMG is observed on the EMG system. Based on the threshold the nerve integrity may be determined. If the threshold is low, then the nerve is intact. If it is high then the nerve might be damaged. If there is no response then the nerve might be severed or completely non-functional. For instance, both of the above are employed in “navigating” in a tumor surgery both in the brain and in the spinal cord.

III An Apparatus of Determining Nerve Proximity and Status

This embodiment of the present invention includes an apparatus comprising a TES test device and a standard EMG processing system. The apparatus is used for determining nerve proximity and status in an open surgical procedure, as well as in a percutaneous surgical procedure. The detailed descriptions of the TES test device of the present invention and the EMG processing system has been fully disclosed above.

### EXAMPLES

The examples provided may be performed using a variety of test device configurations as previously described and set forth.

### Example 1

### A TES Device

The following disclosure represents one of the preferred embodiments, applicable for both open and percutaneous surgical procedures.

The TES is connected to the EMG system via the cable and through the DIN connector and serves as the anode in the montage. A “return” electrode, usually an EEG electrode or needle electrode is placed on the patient flank and serves as the cathode in the montage. The TES is inserted directly into the wound in the open surgical procedure or via an incision, and possibly via a cannula in the percutanous. The TES is placed on the nerve or nerve roots, pedicle screws, or a pedicle screw hole. The operator of the EMG system begins stimulating by increasing the stimulation intensity. The electrical stimulation itself is provided by the EMG system. The stimulation is pulsed at a variable rate, for instance, 2.9 per second, with each pulse having duration of 200 microseconds. This stimulation travels from the EMG system through the cable to the ball tip on the end of the TES. The electrical stimulation is therefore transmitted to the nerve, nerve root, pedicle screw, or pedicle screw hole via the TES. The stimulation level begins at 0 milliamps and is increased until a visible EMG of a minimum of 20 microvolts in amplitude is observed on the EMG system. The threshold is noted.

Different conditions and thresholds means differently. For example, identification of nerve roots and their associated muscles is performed by obtaining thresholds in the about 1 to about 3 milliamp range. Pedicle screws are considered in the correct position if their triggered EMG threshold is greater than 10 milliamps. Pedicle hole breaches are identified by thresholds in the about 2 to about 5 milliamp range.

### Example 2

### Method of Using TES Device for Detecting Nerve Proximity in an Open Surgical Procedure

In one aspect, the TES is used for in an open surgical procedure. A pedicle screw is usually inserted to a target site where an intraoperative X ray is taken for level identification. For instance, for a spinal surgical procedure, introspinous ligaments are removed at the Lumbar (L) 4-5 level when an intraoperative X ray is taken. The remainder of L4 and 5 are identified. Careful dissection is carried out to the transverse processes from L4 to Saccral 1 (S1). An osteotome is then used to take down and remove the facets of L4/5 and L4/S1. The underlying dura is noted and the nerve roots are identified. At this point, a TES is periodically applied to the nerve or nerve roots directly or to the pedicle screw or pedicle screw hole, to identify which nerve fires which muscles or attempt to “label” the nerve roots by differentiating sensory or motor nerve roots.

Furthermore, the nerve roots are decompressed with a Kerrison rongeur and the dura is carefully mobilized. A high-speed bur is then used to decorticate the L4, L5 and S1 pedicles. The starting points are identified and burred. A triple 0 curette is used to find the pedicle followed by ball-tip probe followed by 7.5×40 screws at L4 and L5 and 7.5×38 screws at S1. Triggered EMG thresholds are obtained from each pedicle screw which thresholds are well within normal limits. Intraoperative X rays, anterior/posterior (AP) and lateral, shows good replacement of the screws.

### Example 3

### Method of Using TES Device for Detecting Nerve Proximity in a Percutaneous Surgical Procedure

In another aspect, the TES is used for percutaneous procedures. Such procedures are as follows: intraoperative X rays are taken for level identification. A K wire is inserted via fluoroscopy into the disk space. Following the K wire a cannula is inserted followed by increasing diameters cannulas until a sufficient size is available for the surgical instruments to pass. The diskectomy is performed employing curettes and pituitary rongeur. The diskspace is then dialated employing spacers of increasing size and an interbody device placed within.

Additional x rays are taken to identify the pedicles. A cannula is placed into the wound and “docked” on the pedicle. A K wire is placed via the cannula within the pedicle followed by a tap. A pedicle screw is inserted into the pedicle under fluoroscopy. The pedicle screw is then stimulated employing a triggered EMG test probe via the pedicle and the triggered EMG threshold noted. All thresholds are within the normal range.

The rods are detached to the pedicle screws in the traditional manner. Completed instrumentation x rays demonstrates the pedicles, rods, and interbody fusions in the correct alignment.

### Example 4

### Method of Using the TES Device for Detecting Nerve Status

In another aspect, the TES is used to determine nerve status. A nerve is localized and identified. The TES is placed on the nerve directly and the stimulation intensity ramped from 0 milliamps until a detectable EMG of a minimum 20 microvolts is observed on the EMG system. The threshold is noted. Depending on the threshold, the integrity of the nerve or the nerve functional ability is determined. A low threshold indicates that the nerve is intact and fully functional. A higher threshold indicates that it may be damaged and partially functional. An absent response indicates it is completely non-functional. The nerve may be evaluated anywhere along its length employing the above method any number of times.

### Example 5

### A Test Device Having a Substantially Flush-Tip Electrode

The following disclosure represents one of the preferred embodiments, applicable for both open and percutaneous surgical procedures.

The TES includes at least three components: an electrode used to stimulate the nerves or nerve roots, pedicle screws, or pedicle screw holes during the surgical procedures; means to transmit electric signals from the EMG system to the electrode; and a connector capable of connecting to a standard electromyogram (EMG) processing system to process the electric signals for determination of nerve proximity and status. The electrode is coupled with an insulated shaft of about 6 to 16 inches. The components of the test device are substantially unitary and are operably linked to conduct an electric signal. The components of the substantially unitary device are connected together such that each component will not detach, or not detach easily. The means for connecting the components of the test device, either permanently or semi-permanently, can be any appropriate means depending on the material used for each component of test device. These attaching means include fusing, soldering, gluing, screwing, or any other means that will result in a test device with permanently or semi-permanently fixed components.

The electrode of the TES is substantially flush at its tip, meaning it has a substantially flat tip with a substantially planar surface generally perpendicular to the longitudinal axis thereof. The substantially flat planar surface of the tip of the electrode allows the surgeon to position the TES so as to allow substantial flush contact with the tissue or surface of interest to be stimulated.

The insulating shaft coupled to the electrode is flexible, and preferably capable of reversibly bending. The insulating shaft is malleable or deformable, such that in certain surgical procedures, the surgeon is able to reversibly bend the insulating shaft and adapt the electrode is a desired shape and configuration. When the surgeon places the electrode at a desired location and puts pressure on the test device, the flexible insulating shaft of the test device bends according to the need of the surgeon, and when the surgeon releases the pressure on the test device, preferably the flexible insulating shaft return to its normal and substantially straight shape.

The TES is connected to the EMG system via the cable and through the DIN connector and serves as the anode in the montage. A “return” electrode, usually an EEG electrode or needle electrode is placed on the patient flank and serves as the cathode in the montage. The TES is inserted directly into the wound in the open surgical procedure or via an incision, and possibly via a cannula in the percutanous. The TES is placed on the nerve or nerve roots, pedicle screws, or a pedicle screw hole. The operator of the EMG system begins stimulating by increasing the stimulation intensity. The electrical stimulation itself is provided by the EMG system. The stimulation is pulsed at a variable rate, for instance, 2.9 per second, with each pulse having duration of 200 microseconds. This stimulation travels from the EMG system through the cable to the ball tip on the end of the TES. The electrical stimulation is therefore transmitted to the nerve, nerve root, pedicle screw, or pedicle screw hole via the TES. The stimulation level begins at 0 milliamps and is increased until a visible EMG of a minimum of 20 microvolts in amplitude is observed on the EMG system. The threshold is noted.

Different conditions and thresholds means differently. For example, identification of nerve roots and their associated muscles is performed by obtaining thresholds in the about 1 to about 3 milliamp range. Pedicle screws are considered in the correct position if their triggered EMG threshold is greater than 10 milliamps. Pedicle hole breaches are identified by thresholds in the about 2 to about 5 milliamp range.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified.

What is claimed:

1. A test device comprising a triggered electromyographic stimulator (TES), wherein said TES comprises:
(a) an electrode;
(b) means to transmit signals; and
(c) a connector capable of connecting to a standard electromyogram (EMG) processing system for detecting nerve proximity and status during surgical procedure.

2. The test device of claim 1, wherein said device is for use through a cannula.

3. The test device of claim 1, wherein said device is for use percutaneously.

4. The test device of claim 1, wherein said device is for use directly in open surgical procedures.

5. The test device of claim 1, wherein said pedicle stimulator is substantially straight.

6. The test device of claim 1, wherein said pedicle stimulator comprises a ball electrode.

7. The test device of claim 6, wherein said ball electrode is at one terminus of said device.

8. The test device of claim 6, wherein said ball electrode is about 3.0 mm in diameter.

9. The test device of claim 1, wherein said TES has a length of about 6-16 inches.

10. The test device of claim 1, wherein said means to transmit signals transmits signals from said EMG processing system.

11. The test device of claim 1, wherein said TES is utilized to stimulate the interior of a hole formed in a pedicle or a pedicle screw after insertion into said hole.

12. The test device of claim 1, wherein said TES is utilized to stimulate nerves or nerve roots directly.

13. The test device of claim 11 or 12, wherein said stimulation provides stimulation signals received and processed by said EMG processing system for determination of nerve proximity and status.

14. The test device of claim 1, wherein said EMG processing system is commercially available.

15. A method of determining nerve proximity and status during surgical procedure and assessments, comprising the steps of:
(a) providing a test device of claim 1;
(b) stimulating nerves, nerve roots, or the interior of a hole formed in a pedicle or a pedicle screw after insertion into said hole using said TES;
(c) coupling said TES with said EMG processing system to deliver electrical signals and to receive and process neuromuscular responses due to the stimulation signals; and
(d) determining a nerve proximity and status by identifying relationship between the neuromuscular response and the stimulation signal.

16. The method of claim 15, wherein said test device comprises at least one of a device for maintaining contact with a nerve during surgery, a device for accessing a surgical target site and a device for testing screw placement integrity.

17. The method of claim 15, wherein said EMG processing system determines the nerve proximity between the TES and an existing spinal nerve root to access whether a medial wall of a pedicle has been breached by at least one of hole formation and screw replacement.

18. The method of claim 15, wherein each response is an EMG.

19. The method of claim 15, wherein each response is an EMG measured at a muscle physiologically coupled to the nerve.

20. The method of claim 15, wherein said TES is for use percutanously or in open surgical procedures.

21. An apparatus for determining nerve proximity and status during surgical procedure and assessments, comprising:
(a) a test device of claim 1; and
(b) a standard EMG processing system;
wherein said TES stimulates nerves, nerve roots, or the interior of a hole formed in a pedicle or a pedicle screw after insertion into said hole to produce electrical signals;
wherein said EMG processing system delivers said electrical signals and receives and processes neuromuscular responses due to the stimulation signals; and
wherein a nerve proximity and status is determined by identifying relationship between the neuromuscular response and the stimulation signals.

22. The apparatus of claim 21, wherein said test device comprises at least one of a device for maintaining contact with a nerve during surgery, a device for accessing a surgical target site and a device for testing screw placement integrity.

23. The apparatus of claim 21, wherein said EMG processing system determines the nerve proximity between the TES and an existing spinal nerve root to access whether a medial wall of a pedicle has been breached by at least one of hole formation and screw replacement.

24. The apparatus of claim 21, wherein each response is an EMG.

25. The apparatus of claim 21, wherein each response is an EMG measured at a muscle physiologically coupled to the nerve.

26. The apparatus of claim 21, wherein said apparatus is for use percutaneously or in open procedures.

27. The apparatus of claim 21, wherein said EMG processing system is commercially available.

28. A test device comprising a triggered electromyographic stimulator (TES), wherein said TES comprises:
(a) an electrode comprising a substantially flush tip;
(b) means to transmit signals; and
(c) a connector capable for connecting to a standard electromyogram (EMG) processing system for detecting nerve proximity and status during surgical procedure.
wherein, said components of said TES comprises a unitary flexible test device.

29. The test device of claim 28, wherein said device is for use through a cannula.

30. The test device of claim 28, wherein said device is for use percutaneously.

31. The test device of claim 28, wherein said device is for use directly in open surgical procedures.

32. The test device of claim 28, wherein said pedicle stimulator is substantially straight.

33. The test device of claim 28, wherein said substantially flush tip electrode is at one terminus of said device.

34. The test device of claim 33, wherein said substantially flush tip electrode is about 3.0 mm across.

35. The test device of claim 28, wherein said TES has a length of about 6-16 inches.

36. The test device of claim 28, wherein said means to transmit signals transmits signals from said EMG processing system.

37. The test device of claim 28, wherein said TES is utilized to stimulate the interior of a hole formed in a pedicle or a pedicle screw after insertion into said hole.

38. The test device of claim 28, wherein said TES is utilized to stimulate nerves or nerve roots directly.

39. The test device of claim 37 or 38, wherein said stimulation provides stimulation signals received and processed by said EMG processing system for determination of nerve proximity and status.

40. The test device of claim 28, wherein said EMG processing system is commercially available.

41. A method of determining nerve proximity and status during surgical procedure and assessments, comprising the steps of:
(a) providing a test device of claim 1;
(b) stimulating nerves, nerve roots, or the interior of a hole formed in a pedicle or a pedicle screw after insertion into said hole using said TES;
(c) coupling said TES with said EMG processing system to deliver electrical signals and to receive and process neuromuscular responses due to the stimulation signals; and
(d) determining a nerve proximity and status by identifying relationship between the neuromuscular response and the stimulation signal.

42. The method of claim 41, wherein said test device comprises at least one of a device for maintaining contact with a nerve during surgery, a device for accessing a surgical target site and a device for testing screw placement integrity.

43. The method of claim 41, wherein said EMG processing system determines the nerve proximity between the TES and an existing spinal nerve root to access whether a medial wall of a pedicle has been breached by at least one of hole formation and screw replacement.

44. The method of claim 41, wherein each response is an EMG.

45. The method of claim 41, wherein each response is an EMG measured at a muscle physiologically coupled to the nerve.

46. The method of claim 41, wherein said TES is for use percutanously or in open surgical procedures.
